(19) United States
(12) Patent Application Publication
(10) Pub. No.: US 2025/0000664 A1
(43) Pub. Date: Jan. 2, 2025

(54) CONVERTIBLE GLENOID IMPLANTS

(71) Applicant: HOWMEDICA OSTEONICS CORP., Mahwah, NJ (US)

(72) Inventors: Lance Nathan TERRILL, Cork (IE); David Charles VISCARDI, Glen Rock, NJ (US); Nicholas Kenneth OLSON, Goshen, NY (US)

(73) Assignee: HOWMEDICA OSTEONICS CORP., Mahwah, NJ (US)

(21) Appl. No.: 18/703,819

(22) PCT Filed: Nov. 29, 2022
(86) PCT No.: PCT/US2022/080550

§ 371 Date: Apr. 23, 2024

Related U.S. Application Data
(60) Provisional application No. 63/264,626, filed on Nov. 29, 2021.

(51) Int. Cl. A61F 2/40; A61F 2/30

(52) U.S. Cl. CPC A61F 2/4081; A61F 2002/30138; A61F 2002/305

(57) ABSTRACT

Various embodiments of novel glenoid implant for replacing a portion of an articulation surface of a joint is disclosed. In one example, a prosthetic implant for glenoid includes a baseplate that is configured to be anchored to a bone, and has a bone-facing surface and a second surface opposite the bone-facing surface, where the second surface is configured with a pair of slots for receiving and releasably coupling with a corresponding pair of parallel tabs provided on an articulating component.

### CROSS-REFERENCE TO RELATED APPLICATION

This application claims priority to U.S. Provisional Application No. 63/264,626, filed on Nov. 29, 2021, the entire contents of which are incorporated herein by reference.

### FIELD OF DISCLOSURE

The present disclosure generally relates to glenoid implants for shoulder prosthesis.

### BACKGROUND

A shoulder prosthesis includes a glenoid implant intended to replace the glenoid cavity of the scapula and/or a humeral implant intended to replace the humeral head. The glenoid implant generally includes and articular body intended to articulate with the humeral head, and a fixation means to stabilize the articular body with respect to the scapula.

In many conventional revisable glenoid implants utilizing metal baseplate with polymer liners, the primary glenoid implants are revised to replace the polymer liner or to substitute the liner with a reverse prosthesis glenosphere. Such revision procedures often results in a lot of bone loss which is not desired. Therefore a reliable convertible glenoid implant is desired.

### SUMMARY

Provided herein are various embodiments of implants that are useful in total or partial shoulder arthroplasty. According to an aspect, a prosthetic implant for glenoid is disclosed. The implant comprises a baseplate that is configured to be anchored to a bone. The baseplate comprises a bone-facing surface and a second surface opposite the bone-facing surface, where the second surface is configured with a pair of slots for receiving and releasably coupling with a corresponding pair of parallel tabs provided on an articulating component.

Also disclosed is a prosthetic implant for glenoid that comprises a bearing component that includes a bearing surface, a baseplate-facing surface, and a pair of parallel tabs provided on the baseplate-facing surface; and a baseplate configured to be anchored to a bone, and comprising a bone-facing surface and a second surface opposite the bone-facing surface, wherein the second surface is configured with a pair of parallel slots for receiving and engaging with the corresponding pair of parallel tabs for releasably coupling the bearing component to the baseplate.

Also provided is a prosthetic implant system for glenoid. The implant system comprises a baseplate configured to be anchored to a bone, and comprising a bone-facing surface and a second surface opposite the bone-facing surface, wherein the second surface is configured with a pair of parallel slots for receiving and releasably coupling with a bearing component or a glenosphere component; a bearing component that comprises a bearing surface, a baseplate-facing surface, and a pair of parallel tabs provided on the baseplate-facing surface configured to engage with the pair of parallel slots on the baseplate to form the releasable coupling with the baseplate; and a glenosphere component that comprises glenosphere surface, a baseplate-facing surface, and a pair of parallel tabs provided on the baseplate-facing surface configured to engage with the pair of parallel slots on the baseplate to form the releasable coupling with the baseplate.

In another embodiment of a glenoid implant, the implant comprises a body having an articulation surface and a bone-facing surface opposite the articulation surface, wherein the articulation surface is formed of a polymer; wherein the bone-facing surface comprises a central portion that is formed of a metal and a peripheral portion that is integrally formed of the polymer along with the articulation surface; and wherein the central portion includes a fixation post extending from bone-facing surface, the fixation post comprising one or more graft holes extending through the fixation post along an anterior-posterior direction.

In another embodiment of a glenoid implant, the implant comprises a body having an articulation surface and a bone-facing surface opposite the articulation surface, wherein the articulation surface is formed of a polymer; wherein the bone-facing surface comprises a central portion that is formed of a metal and a peripheral portion that is integrally formed of the polymer along with the articulation surface; and wherein the central portion includes a fixation post extending from bone-facing surface and one or more fins extending from the bone-facing surface.

In another embodiment of a glenoid implant, the implant comprises a body having an articulation surface and a bone-facing surface opposite the articulation surface, wherein the articulation surface is formed of a polymer; wherein the bone-facing surface comprises a central portion that is formed of a metal and a peripheral portion that is integrally formed of the polymer along with the articulation surface; and wherein the central portion includes a fixation post extending from bone-facing surface and a plurality of spikes extending from the bone-facing surface.

In another embodiment of a glenoid implant, the implant comprises a body having an articulation surface and a bone-facing surface opposite the articulation surface, wherein the articulation surface is formed of a polymer; wherein the bone-facing surface comprises a central portion that is formed of a metal and a peripheral portion that is integrally formed of the polymer along with the articulation surface; wherein the central portion protrudes from the peripheral portion, thereby forming a stepped edge along the perimeter of the bone-facing surface; and wherein the central portion includes a fixation post extending from bone-facing surface.

### BRIEF DESCRIPTION OF THE DRAWINGS

The various embodiments of the inventive implants of the present disclosure will be described in more detail in conjunction with the following drawing figures. The structures in the drawing figures are illustrated schematically and are not intended to show actual dimensions.

FIG. 1A is an isometric exploded view of a glenoid implant according to an embodiment of the present disclosure showing a glenoid bearing component and a convertible baseplate component.

FIG. 1B is an isometric view of the convertible baseplate showing its second side that is configured to receive an articulation component.

FIG. 1C is a sectional view of the convertible baseplate.

FIG. 1D is an isometric view of a glenoid bearing component's baseplate-facing surface.

FIG. 1E is a sectional view of an assembled glenoid implant according to an embodiment where the glenoid bearing component is coupled to the convertible baseplate of the present disclosure.

FIG. 1F is a sectional view of the glenoid bearing component and the convertible baseplate that are in the process of being fully coupled.

FIG. 1G is an isometric view of the bone-facing surface side of the assembled glenoid implant of FIGS. 1A and 1E.

FIG. 1H is a side view of the assembled glenoid implant of FIG. 1G.

FIG. 1I is an illustration of another embodiment of the glenoid implant of the present disclosure in which the glenoid bearing component and the baseplate are configured to engage each other by sliding.

FIGS. 2A-2C are illustrations of glenosphere version of the articulation component of the glenoid implant of the present disclosure.

FIGS. 2D-2F are illustrations of another embodiment of the glenosphere version of the articulation component of the glenoid implant of the present disclosure.

FIGS. 3A-3C are illustrations of an embodiment of a glenoid bearing component version of the articulation component of the glenoid implant of the present disclosure.

FIGS. 4A-4C are illustrations of another embodiment of a glenoid bearing component version of the articulation component of the glenoid implant of the present disclosure.

FIGS. 5A-5B are illustrations of another embodiment of a glenoid bearing component version of the articulation component of the glenoid implant of the present disclosure.

FIGS. 6A-6C are illustrations of another embodiment of a glenoid bearing component version of the articulation component of the glenoid implant of the present disclosure.

FIGS. 7A-7C are illustrations of an embodiment of a glenoid bearing implant according to the present disclosure.

FIGS. 8A-8B are illustrations of another embodiment of a glenoid bearing implant according to the present disclosure.

FIGS. 9A-9B are illustrations of another embodiment of a glenoid bearing implant according to the present disclosure.

FIG. 10 is illustrations of another embodiment of a glenoid bearing implant according to the present disclosure.

FIGS. 11A-11C are illustrations of an embodiment of a configuration for asymmetrically arranged fixation elements for attaching a glenoid bearing implant or a baseplate to a glenoid when the desired target anchoring point in the glenoid is asymmetrically located.

FIG. 12 is an illustration of a glenoid bearing implant according to another embodiment showing a configuration for a primary fixation of the implant.

FIGS. 13A-13B are illustrations of another embodiment of a glenoid bearing implant showing another configuration for a primary fixation of the implant.

FIGS. 14A-14B are illustrations of another embodiment of a glenoid bearing implant showing another configuration for a primary fixation of the implant.

FIG. 15 is an illustration of another configuration of fixation elements for achieving enhanced primary fixation of a baseplate implant to a glenoid.

FIG. 16 is an illustration of another configuration for achieving enhanced primary fixation of a glenoid bearing implant.

FIG. 17 is an illustration of a new bone screw construct that would be useful in glenoid implant applications.

FIG. 18 is an illustration of an embodiment of a baseplate for reverse shoulder arthroplasty (RSA) application.

FIGS. 19A-19B are illustrations of another embodiment of a baseplate for RSA application.

FIG. 20 is an illustration of another embodiment of a baseplate for RSA application.

FIGS. 21A-21D are illustrations of an embodiment of a baseplate configured for modular augments for RSA application.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Provided herein are various improved glenoid implants that have articulation surface that is configured to engage with an anatomical humeral head or a humeral component of a shoulder replacement implant system. Therefore, references to “a humeral head” as used herein should be construed to include both an anatomical humeral head as well as implant humeral head.

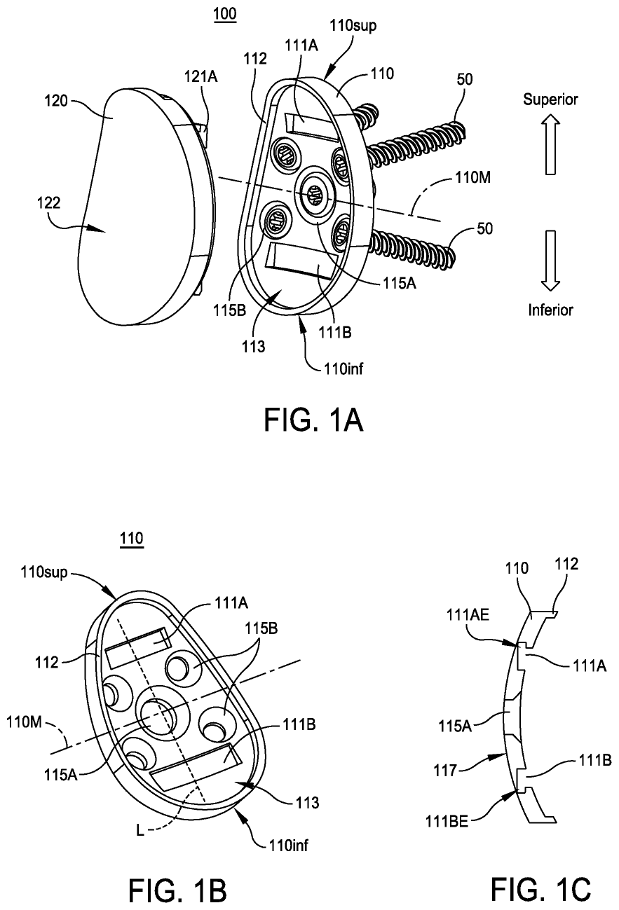

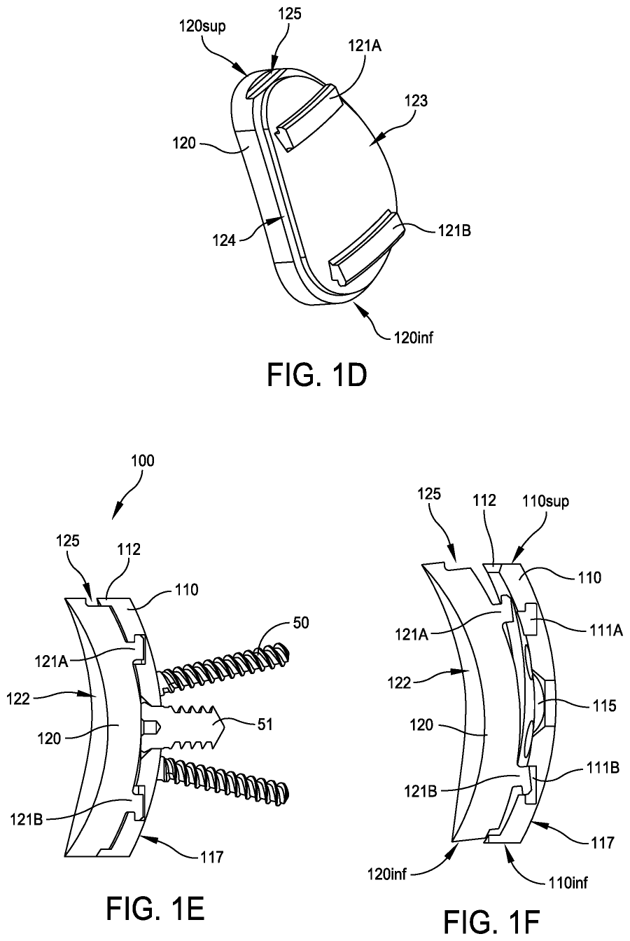

Referring to FIGS. 1A-IF, a prosthetic implant 100 for glenoid according to an embodiment is disclosed. In one aspect, the implant 100 comprises a baseplate 110 and an articulating component. The baseplate 110 is configured to be anchored to a bone, in this case a glenoid, and comprises a bone-facing surface 117 and a second surface 113 opposite the bone-facing surface 117. The second surface 113 is configured with a pair of slots 111A, 111B for receiving and releasably coupling with the articulating component, where the articulating component is configured with a corresponding pair of tabs 121A, 121B that engage with the slots 111A, 111B. The articulating component can be a bearing component 120, as shown in FIGS. 1A-IF, that replaces the articulating bearing surface of a glenoid in an anatomic prosthetic configuration or it can be a glenosphere component 200, 200′, as shown in FIGS. 2A-2F, in a reverse prosthetic configuration.

The baseplate 110 is shaped to generally conform to the anatomical shapes and contours of glenoid to enhance the securement of the baseplate 110 to a glenoid and enhance the durability of the prosthesis implant in a patient. Therefore, the shape of the baseplate 110 is configured to have a superior end and an inferior end corresponding to the glenoid's anatomical direction.

In FIGS. 1A and 1B, the anatomical superior direction and inferior direction are noted. The corresponding superior end and the inferior end of the baseplate 110 are designated as Sup and Inf, respectively. A median line 110M is defined halfway between the superior end 110sup and the inferior end 110inf. One of the pair of slots 111A is located on the superior end 110sup side of the median line 110M and the other of the pair of slots 111B is located on the inferior end 110inf side of the median line 110M.

In some embodiments, the slot 111A located on the superior end side of the median line 110M is located closer to the superior end 110sup than the median line 110M and the slot 111B located on the inferior end 110inf side of the median line is located closer to the inferior end 110inf than the median line 110M. This configuration maximizes the distance between the two slots 111A and 111B forming the releasable coupling between the baseplate and the articulating component to enhance the stability of the coupling by minimizing any rocking motion between the baseplate and the articulating component in the superior-inferior direction.

In some embodiments, each of the pair of slots 111A, 111B can be shaped so that their outlines have a curvature. In some embodiments, the baseplate 110 comprises a rim 112 along the periphery of the second surface 113 where the rim extends away from the bone-facing surface 117 and the curvature of the slot 111A located on the superior end 110sup side of the median line 110M is substantially parallel to the curvature of the rim 112 along the superior end of the baseplate 110 and the curvature of the slot 111B located on the inferior end 110inf side of the median line is substantially parallel to the curvature of the rim 112 along the inferior end of the baseplate. In other words, the curvature of the slot 111A follows the curvature of the rim 112 along the superior end 110sup of the baseplate 110 and the curvature of the slot 111B follows the curvature of the rim 112 along the inferior end 110inf of the baseplate.

In some embodiments, the curvature of the slot 111A located on the superior end side of the median line 110M has a radius of curvature that is not less than radius of curvature of the rim along the superior end 110sup, and the curvature of the slot 111B located on the inferior end 110inf side of the median line 100M has a radius of curvature that is not less than radius of curvature of the rim along the inferior end 110inf.

In some embodiments, each of the pair of slots 111A, 111B are straight and they are parallel to each other and to the median line 110M. In some embodiments, one of the pair of slots is located on the superior end 110sup side of the median line 100M and the other of the pair of slots is located on the inferior end 110inf side of the median line.

Referring to FIG. 1C, which is an illustration showing a longitudinal section view of a baseplate 110, each of the pair of parallel slots 111A, 111B has an L-shaped a cross-section having an elbow portion 111AE and 111BE, respectively. The elbow portions 111AE and 111BE of the L-shape of the slots point away from each other. In FIG. 1D, which is an isometric view of the bearing component 120, the ends of the corresponding parallel tabs 121A and 121B can be seen with the corresponding L-shaped cross-sectional profile. The L-shape of the tabs 121A and 121B also have elbow portions 121AE and 121BE.

FIG. 1F is a sectional view of a bearing component 120 and a baseplate 110 and shows the preferred way of coupling the bearing component 120 to the baseplate 110. The bearing component 120 is brought together with the baseplate 110 with the inferior end 120inf of the bearing component 120 is brought together with the inferior end 110inf of the baseplate 110 first so that the tab 121B of the bearing component 120 is aligned and inserted into the slot 111B of the baseplate 110. Then, bearing component 120 is pivoted about the inferior end 120inf toward the baseplate 110 so that the tab 121A on the superior end 120sup of the bearing component 120 engages the corresponding slot 111A of the baseplate 110. This engaging motion pushes the tab 121A into the slot 111A to form a snap-fit engagement. The elbow portion 121AE of the tab 121A and the elbow portion 111AE of the slot 111A help form the snap-fit engagement.

As shown in FIGS. 1A and 1E, the second surface 113 of the baseplate 110 that engages the bearing component 120 is recessed where the recess is formed by a ridge 112 that extends around the periphery of the baseplate 110. The ridge 112 can be a continuous structure that fully circumscribes baseplate 110 or in some embodiments, the ridge 112 can have one or more breaks in it as appropriate. In some embodiments, the bearing component 120 can be sized to fit within the perimeter defined by the ridge 112. In some embodiments, as shown in FIG. 1D, the baseplate facing surface 123 of the bearing component 120 can have a ledge 124 along the periphery that conforms to the ridge 112 so that the baseplate facing surface 123 sits within the recess formed by the ridge 112. In these embodiments, the ridge 112 enhances the stability of the coupling between the bearing component 120 and the baseplate 110 by surrounding the bearing component 120.

The bearing component 120 can be made from a high modulus polymer. As used herein, “high modulus polymer” for any of the implant components refer to such materials as, for example, polyethylene (e.g. ultra-high-molecular-weight polyethylene (UHMWPE)), polyether ether ketone (PEEK), etc. All references to UHMWPE herein includes all variants of UHMWPE in orthopedic application such as vitamin E diffused UHMWPE. In some embodiments, the bearing component can be made from hydrogel material, a three-dimensional solid resulting from cross-linked hydrophilic polymer chains formed of polyvinyl alcohol (PVA). The baseplate 110 can be made from a material that is more structurally rigid, such as metal.

To help disassemble the bearing component 120 from the baseplate 110, a notch 125 is provided on the superior end 120sup of the bearing component 120. As shown in the sectional view in FIG. 1E, the notch 125 provides a gap between the bearing component 120 and the top of the ridge 112 of the baseplate 110. A suitable tool can be inserted into the gap provided by the notch 125 to pry the superior end 120sup of the bearing component 120 away from the baseplate 110 and pull the tab 121A out of the slot 111A. Once the tab 121A is pulled out of the slot 111A, the bearing component 120 can be pivoted about its inferior end 120inf away from the baseplate 110. The notch 125 on the superior end of the bearing component 120 provides allows disassembly without damaging the fixation of the baseplate 110 to the glenoid.

The baseplate 110 can also comprise a plurality of countersunk holes for receiving fixation elements such as bone screws/pegs for securing the baseplate 110 to the glenoid. In the illustrated example, the baseplate 110 has five countersunk holes: one centrally located hole 115A and four more peripherally located holes 115B. The diameter of the holes can be selected to accommodate the particular set of bone screws intended for use. In the illustrated example, the centrally located hole 115A has a larger diameter than the other holes. Because of the geometry of the glenoid bone, the centrally located hole 115A can be configured to accommodate a larger diameter bone screw and the more peripherally located holes 115B can be configured to accommodate smaller diameter bone screws.

In some embodiments, the bone-facing surface of the baseplate 110 can further comprise an ongrowth or ingrowth surface material to provide additional stability for the fixation of the baseplate 110.

According to an aspect of the present disclosure, a prosthetic implant 100 for glenoid comprises a baseplate 110 and an articulating component that is a bearing component 120. The bearing component 120 comprises a bearing surface 122, a baseplate-facing surface 123, and a pair of tabs 121A and 121B provided on the baseplate-facing surface 123. The pair of tabs 121A, 121B are configured and located so that they can be received by and engage with the corresponding pair of slots 111A, 111B for releasably coupling the bearing component 120 to the baseplate 110.

The bearing component 120 has generally the same outline shape as the baseplate 110 and comprises a superior end 120sup and an inferior end 120inf corresponding to the glenoid's anatomical directions. Accordingly, the superior end 120sup and the inferior end 120inf of the bearing component 120 align with the superior end 110sup and inferior end 110inf of the baseplate 110 when the two components are engaged. As with the baseplate 110, a median line 120M can be defined for the bearing component 120 halfway between the superior end 120sup and the inferior end 120inf.

One of the pair of tabs 121A is located on the superior end 120sup side of the median line 120M at a location so that the tab 121A aligns with the slot 111A on the superior end 110sup side of the baseplate 110. The other of the pair of tabs 121B is located on the inferior end 120inf side of the median line 120M at a location so that the tab 121B aligns with the slot 111B on the inferior end 110inf side of the median line 120M.

As the configuration of the pair of tabs 121A and 121B mirror the configuration of the pair of slots 111A and 111B on the baseplate 110, the tab 121A located on the superior end 120sup side of the median line 120M is located closer to the superior end 120sup than the median line 120M and the slot 121B located on the inferior end 120inf side of the median line 120M is located closer to the inferior end 120inf than the median line 120M.

In some embodiments, the bearing component 120 is configured so that one of the pair of tabs 121A and 121B on the bearing component 120 is configured to form a snap-fit engagement with the corresponding one of the pair of slots 111A, 111B on the baseplate 110. In some embodiments, the tab 121A located near the superior end 120sup of the bearing component 120 is configured to form a snap-fit engagement with the corresponding slot 111A that is located on the superior end 110sup side of the median line on the baseplate 110.

In some embodiments, the pair of tabs 121A, 121B have curved outline to couple with the embodiment of the baseplate 110 that has slots 111A, 111B that have curved outlines.

In some embodiments, the pair of tabs 121A, 121B have straight outlines and are parallel to each other and to the median line 120M to couple with the embodiment of the baseplate 110 that has slots 111A, 111B that have straight outlines and are parallel to each other and to the median line 110M.

FIG. 1G shows an isometric view of the assembled baseplate 110 and the bearing component 120 with bone screws 50, 51. FIG. 1H shows a side view of the assembly shown in FIG. 1G.

In view of the anatomic directions of the glenoid, two sides of the baseplate 110 can be identified as an anterior side 110ant and a posterior side 110pos corresponding to the glenoid's anatomical directions. Referring to FIG. 1I, in some embodiments, the pair of slots 111A′ and 111B′ on the baseplate 110 are straight, oriented parallel to each other, and open to one side, either the anterior side 110ant or the posterior side 110pos of the baseplate 110 such that the bearing component 120 can releasably couple with the baseplate 110 by sliding the corresponding pair of parallel tabs 121A and 121B into the pair of parallel slots 111A′ and 111B′ from the side that is open. In FIG. 1I, the bearing component 120 is shown from the bearing surface side so that the tabs 121A, 121B which are on the opposite surface are illustrated with phantom lines. The baseplate-facing surface 123 of the bearing component 120 and the second surface 113 of the baseplate can be configured to form an interference fit when the bearing component is fully seated in the baseplate, thereby preventing the bearing component from unintentionally being disengaged by sliding back out. For example, a protuberance such as a nub 119 can be provided on the second surface 113 of the baseplate 110 and a corresponding recess 129 can be provided on the baseplate-facing surface 123 of the bearing component 120 so that they form an interference fit.

In some embodiments, a prosthetic implant system for glenoid is disclosed. Such system comprises the baseplate 110 described herein, the bearing component 120 described herein, and a glenosphere component. Some examples of the glenosphere component 200 and 200′ are illustrated in FIGS. 2A-2F.

Referring to FIG. 2A, the glenosphere component 200 embodiment comprises a glenosphere 130G providing a convex (e.g., spherical) articular surface, and a baseplate-engaging portion 130. The baseplate-engaging portion 130 includes a baseplate-facing surface 133 and a pair of parallel tabs 131A, 131B provided on the baseplate-facing surface 133. The tabs 131A and 131B are configured to engage with the corresponding pair of parallel slots 111A, 111B on the baseplate 110 to form the releasable coupling with the baseplate.

The baseplate-engaging portion 130 is structures similar to the bearing component 120 in terms of the structures involved in the releasable coupling with the baseplate 110. Rather than the bearing surface 122, however, the baseplate-engaging portion 130 has a glenosphere 130G.

FIGS. 2B and 2C are a perspective view and a side view, respectively, of the glenosphere component 200 that is assembled with the baseplate 110, including the bone screws 50 and 51.

FIG. 2D shows a glenosphere component 200′ according to another embodiment. The glenosphere component 200′ also comprises a glenosphere 130G′, a baseplate-engaging portion 130, a baseplate-facing surface 133 on the baseplate-engaging portion 130, and a pair of parallel tabs 131A, 131B provided on the baseplate-facing surface 133 configured to engage with the pair of parallel slots 111A, 111B on the baseplate 110 to form the releasable coupling with the baseplate. The structure of the baseplate-engaging portion 130 as far as the structures involved in forming the releasable coupling with the baseplate 110 are same as those described above for the bearing component 120. The baseplate-engaging portion 130 can be made of the same material as the bearing component 120 discussed above. For example, the baseplate-engaging portion 130 can be made of molded PEEK.

FIGS. 2D and 2F are a perspective view and a side view, respectively, of the glenosphere component 200′ that is assembled with the baseplate 110, including the bone screws 50 and 51. The glenosphere 130G′ of the glenosphere component 200′ has a larger diameter than the glenosphere 130G of the glenosphere component 200 which may be more appropriate in certain patients. Because the glenosphere 130G′ has a larger diameter, the glenosphere can include a skirt portion 130G'S that extend toward the glenoid. FIG. 2F is an illustration of a cross-sectional view (section taken along a plane lying in the anterior-posterior direction) of the implant system of the glenosphere component 200′ and the baseplate 110 implanted onto a prepared glenoid. The diameter of the glenosphere 130G′ is larger than the span of the glenoid so that the skirt portion 130G'S encompasses the glenoid. As shown in FIGS. 2D and 2E, the skirt portion 130G'S has cutouts 138sup and 138inf in the superior direction and the inferior direction, respectively, to accommodate for the superior-inferior movement of the shoulder joint.

As with the bearing component 120, the glenosphere components 200, 200′ are configured such that one of the pair of parallel tabs 131A, 131B on the glenosphere components 200, 200′ forms a snap-fit engagement with the corresponding one of the parallel slots 111A, 111B on the baseplate. In some embodiments, the superiorly located tab 131A on the glenosphere components 200, 200′ forms the snap-fit engagement.

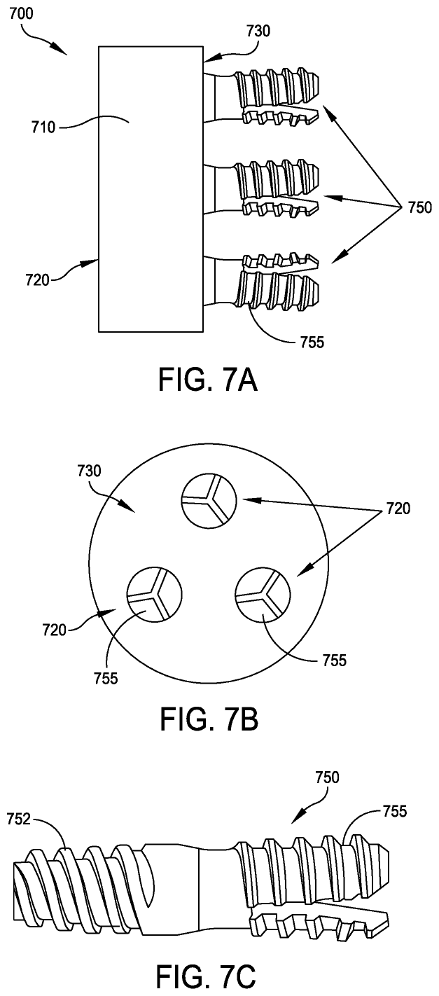

In some embodiments, the baseplate 110 of the prosthetic implant system is configured to engage the articulating components (the bearing component 120 and glenosphere component 200, 200′) in a sliding manner as the embodiment described above in reference to FIG. 11.

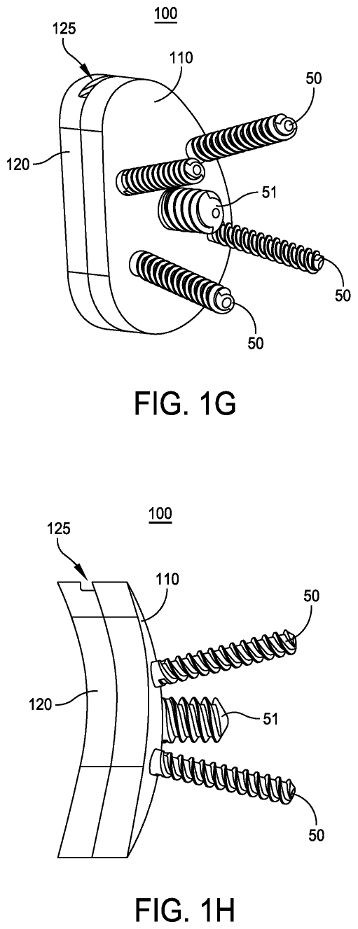

Referring to FIGS. 3A-3C, disclosed is a glenoid implant 300 having a hybrid post structure. The glenoid implant 300 includes a body 310 having an articulation surface 320 and a bone-facing surface 330 opposite the articulation surface 320. The articulation surface 320 formed of a polymer. The bone-facing surface 330 comprises a central portion 332 that is formed of a metal and a peripheral portion 335 that is formed of a polymer. The peripheral portion 335 is integrally formed of the polymer along with the articulation surface 320. The central portion 332 includes a fixation post 333 extending from the bone-facing surface 330.

The fixation post 333 anchors the glenoid implant 300 in the scapula that has been prepared with an appropriately sized hole to receive the fixation post 333. The fixation post 333 comprises one or more graft holes extending, orthogonal to the longitudinal axis 333L of the fixation post 333, through the fixation post along an anterior-posterior A-P direction. In the illustrated example, one such graft hole 337H is shown. The graft hole 337H is provided to accommodate a bone graft material when the glenoid implant 300 is implanted.

In some embodiments, the glenoid implant 300 further comprises a plurality of fixation elements 337 extending from the peripheral portion of the bone-facing surface. In some embodiments, the plurality of fixation elements 337 are posts configured with one or more grooves 337G for holding a quantity of bone cement.

In some embodiments, the central portion 332 including the fixation post 333 can be configured to have a porous or textured surface that can promote bone tissue ongrowth. In some embodiments, the central portion 332 including the fixation post 333 can be made of 3-D porous material, such as Stryker's Tritanium® or Wright Medical Technology's ADAPTIS™, that can promote bone tissue ongrowth/ingrowth. Either option provides enhanced bonding between the implant 300 and the bone. Throughout this disclosure, the surface of such component will be referred to as “a textured surface.”

The central portion 332 can be an insert formed of metal and a high modulus polymer or hydrogel can be molded onto the metal insert to form the body 310.

In some embodiments, the fixation post 333 has a necked portion 333N that has a smaller diameter. The necked portion 333N allows easier insertion of the fixation post 333 into a prepared glenoid surface.

To implant the glenoid implant 300 onto a glenoid, the glenoid bone surface is prepared to accommodate the contour of the bone-facing surface 330. Then holes are drilled into the glenoid to receive the fixation post 333 and fixation elements 337.

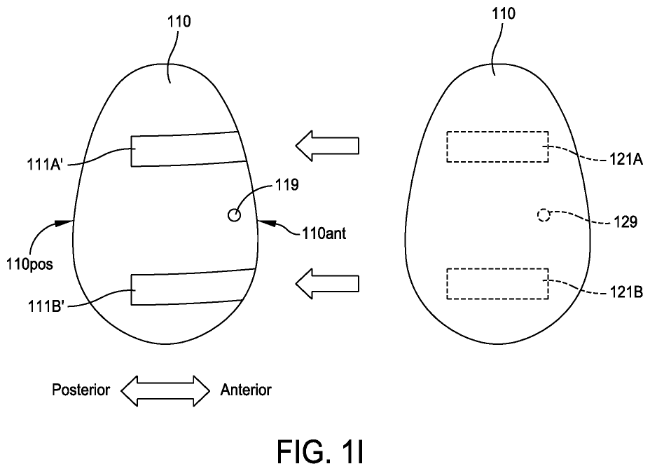

Referring to FIGS. 4A-4C, disclosed is a glenoid implant 400 having a hybrid post structure with fins. The glenoid implant 400 comprises a body 410 having an articulation surface 420 and a bone-facing surface 430 opposite the articulation surface 420. The articulation surface 420 is formed of a polymer. The bone-facing surface 430 comprises a central portion 432 that is formed of a metal and a peripheral portion 435 that is integrally formed of the polymer along with the articulation surface 420. The central portion 432 includes a fixation post 433 extending from bone-facing surface 430 and one or more fins 450 extending from the bone-facing surface 430.

In some embodiments, the glenoid implant 400 further comprises a plurality of fixation elements 437 extending from the peripheral portion 435 of the bone-facing surface 430. The plurality of fixation elements 437 are posts configured with one or more grooves 437G for holding a quantity of bone cement.

In some embodiments, the central portion 432 has a textured surface that can promote bone tissue ongrowth/ingrowth. In some embodiments, the one or more fins 450 also have a textured surface that can promote bone tissue ongrowth/ingrowth.

The central portion 432 can be an insert formed of metal and a high modulus polymer or hydrogel can be molded onto the metal insert to form the body 410 including the articulation surface 420 and the peripheral portion 435.

In some embodiments, the fixation post 433 has a necked portion 433N that has a smaller diameter. The necked portion 433N allows easier insertion of the fixation post 433 into a prepared glenoid surface.

To implant the glenoid implant 400 onto a glenoid, the glenoid bone surface is prepared to accommodate the contour of the bone-facing surface 430. Then holes are drilled into the glenoid to receive the fixation post 433 and fixation elements 437.

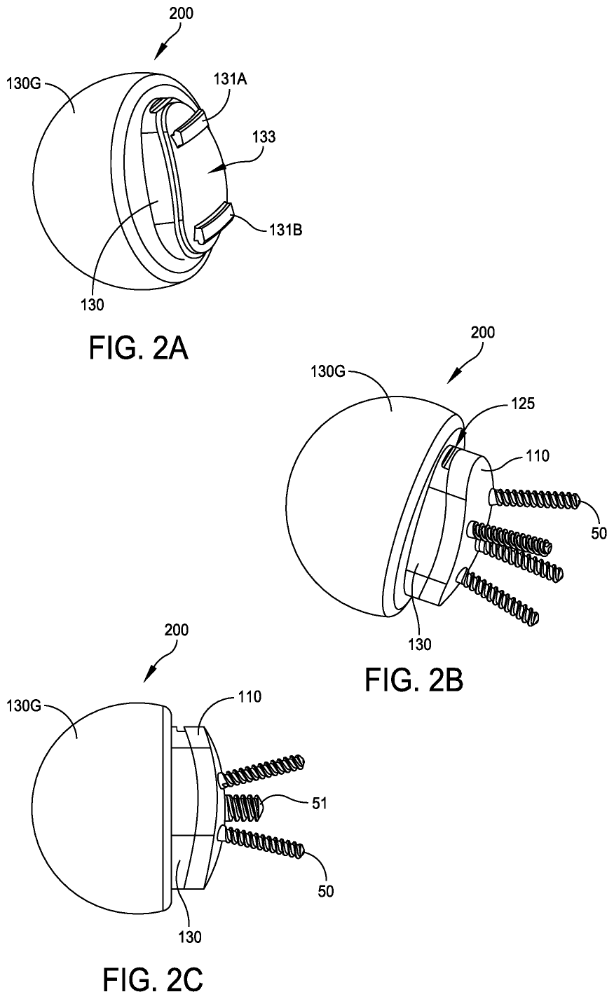

Referring to FIGS. 5A-5C, disclosed is a glenoid implant 500 having a hybrid post structure with spikes. The glenoid implant 500 comprises a body 510 having an articulation surface 520 and a bone-facing surface 530 opposite the articulation surface. The articulation surface 520 is formed of a polymer. The bone-facing surface 530 comprises a central portion 532 that is formed of a metal and a peripheral portion 535 that is integrally formed of the polymer along with the articulation surface 520.

The central portion 532 includes a fixation post 533 extending from bone-facing surface 530 and a plurality of spikes 537 extending from the bone-facing surface 530.

When implanted into a patient, the spikes get fixed into punched or drilled holes in the prepared glenoid surface. The spikes are generally smaller than fixation posts or pegs and thus can be provided on the central portion 532 close to the outer perimeter of the implant which provides more secure attachment of the implant 500 to the glenoid and reduce micromotion in terms of superior-inferior rocking.

In some embodiments, the fixation post 533 can comprise one or more annular grooves 533G for holding a quantity of bone cement.

In some embodiments, the central portion 532 has a textured surface that can promote bone tissue ongrowth/ingrowth. The central portion 532 can be an insert formed of metal and a high modulus polymer or hydrogel can be molded onto the metal insert to form the body 510 including the articulation surface 520 and the peripheral portion 535.

In some embodiments, the plurality of spikes 537 can also have a textured surface that can promote bone tissue ongrowth/ingrowth.

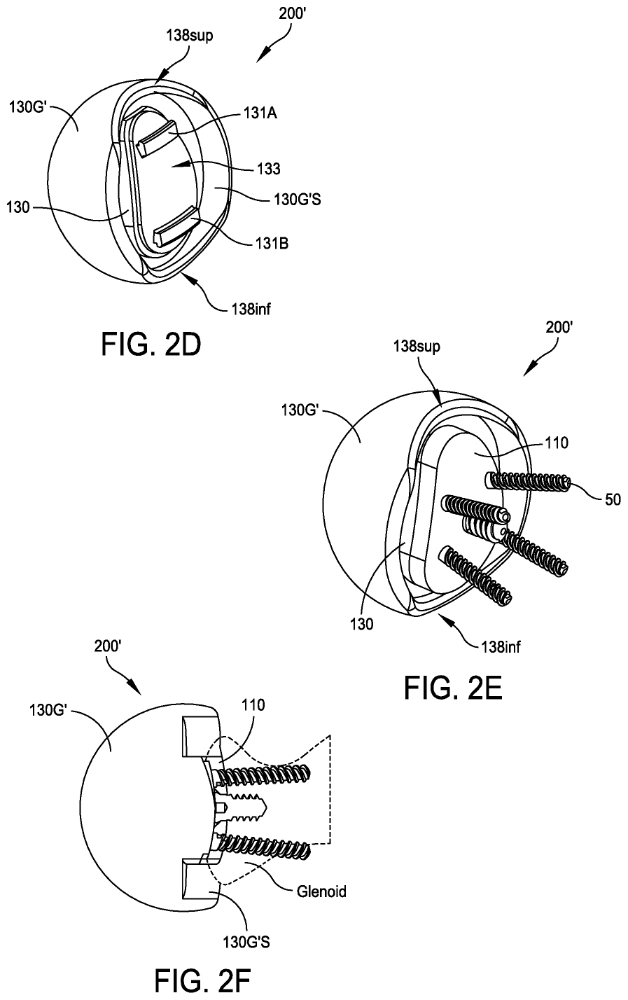

Referring to FIGS. 6A-6C, disclosed is a glenoid implant 600 configured with an inset portion that engages with a prepared glenoid. The glenoid implant 600 comprises a body 610 having an articulation surface 620 and a bone-facing surface 630 opposite the articulation surface. The articulation surface 620 is formed of a polymer. The bone-facing surface 630 comprises a central portion 632 that is formed of a metal and a peripheral portion 635 that is integrally formed of the polymer along with the articulation surface 620.

The central portion can be an insert formed of metal and a high modulus polymer or hydrogel can be molded onto the metal insert to form the body 610 including the articulation surface 620 and the peripheral portion 635.

The central portion 632 protrudes from the peripheral portion, thereby forming a stepped edge E along the perimeter of the bone-facing surface. The central portion 632 has an outline that is smaller than the outline of the body 610 defined by the peripheral portion thus the central portion 632 and the peripheral portion 635 form the stepped edge E. This can be better seen in the side view shown in FIG. 6B. To implant the glenoid implant 600 onto a glenoid, a recess that complements the shape and outline of the protruding central portion 632 is formed by reaming the glenoid surface. A robotic reamer can be used to perform such reaming procedure. The glenoid implant 600 is formed to have an outline that matches the outline of the glenoid surface. Thus, when the protruding central portion 632 is inset into the reamed recess in the glenoid, the polymer peripheral portion 635 of the stepped edge E rests on the cortical bone. This is illustrated in FIG. 6C.

The central portion 632 can include a fixation post 633 extending from bone-facing surface 630. The fixation post 633 can comprise one or more annular grooves 633G for holding a quantity of bone cement.

In some embodiments, the central portion 632 has a textured surface that can promote bone tissue ongrowth/ingrowth.

### [Alternative Fixation Constructs]

Also disclosed are various alternative constructs for establishing primary fixation of glenoid implant to the glenoid.

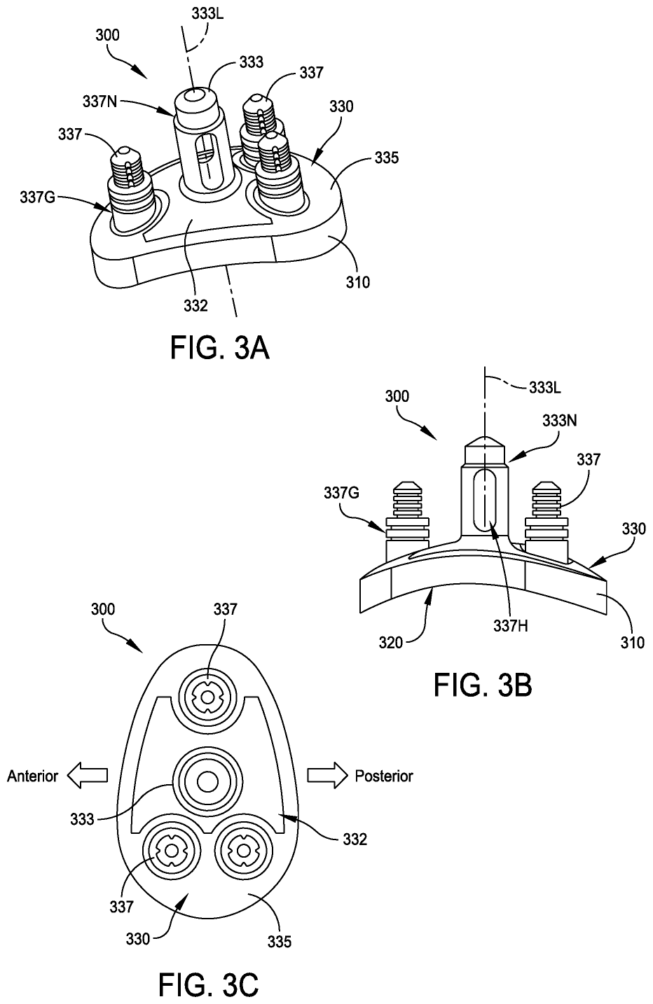

Referring to FIGS. 7A-7C, an embodiment of glenoid bearing implant 700 comprises a body 710 having a plurality of interference-fitting fixation elements 750 extending from the bone-facing surface 730 of the implant. An articulation surface 720 is on the opposite side from the bone-facing surface. An example of the interference-fitting fixation elements 750 is shown in FIG. 7C. The interference-fitting fixation element 750 has the same structure as Stryker's ToeTac® hammer toc fixation implants and comprises a first end 752 that is configured to be threaded into the bone-facing surface 730 of the implant body 710, and a second end 755 that extends out from the bone-facing surface 730 for engaging into a glenoid to secure the implant 700.

The second end 755 is configured to be inserted into a hole prepared into the prepared surface of a glenoid and resist backing out. As shown in FIG. 7B which is a plan view of the bone-facing surface 730, the second end 755 of the interference-fitting fixation element 750 is divided into two or more (three shown in the example) legs that are flared radially outward in their at rest state. To secure the implant 700 to a glenoid, the second end 755 of the interference-fitting fixation element 750 is forced into a hole prepared in the glenoid. The diameter of the prepared hole in the glenoid is such that the radially flared legs of the fixation element 750 gets compressed radially inward as the fixation element 750 enters the hole. The resilience of the legs urges the legs radially outward. As shown in FIG. 7C, the outer surface of the radially flared legs forming the second end 755 are configured with ridges so that when the legs are urged against the inside wall of the hole in the glenoid, the ridges bite into the bone forming an interference fit and prevent the fixation element 750 from backing out of the hole. The fixation elements 750 are made from a resilient material such as PEEK.

Referring to FIG. 7B, the interference-fitting fixation elements 750 are arranged on the bone-facing surface 730 as close to the periphery as possible given the spatial restraint imposed by the geometry of the metaphysical region of the glenoid. This arrangement maximizes the stability of the implant-glenoid bond and minimize rocking motion of the body 710 after being installed.

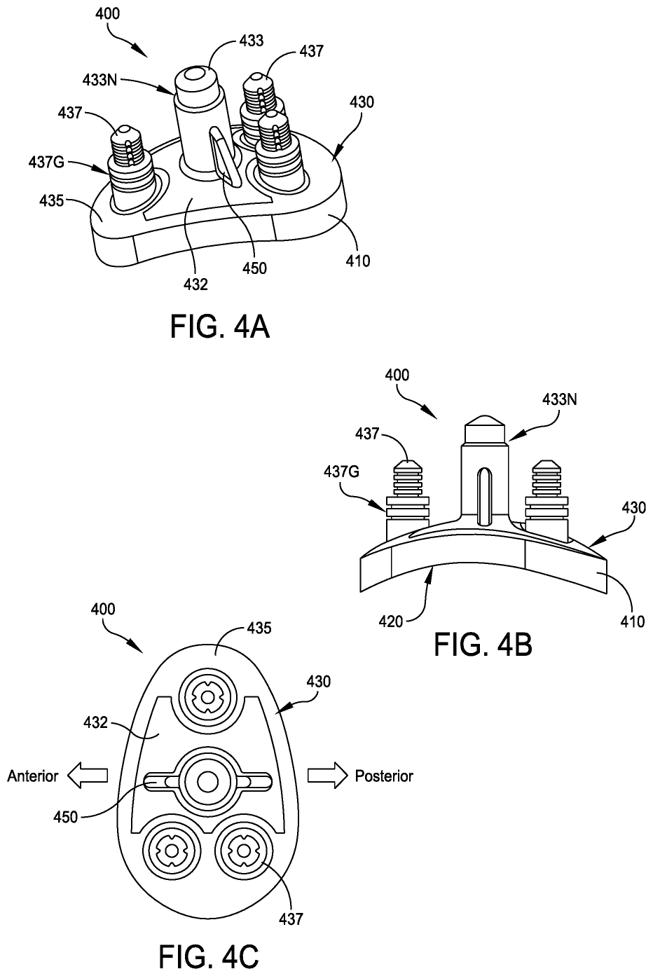

FIGS. 8A-8B show a glenoid bearing implant 800 according to another embodiment. The implant 800 comprises a body 810 having a plurality of fixation elements 850, 860 extending from the bone-facing surface 830 of the implant. An articulation surface 820 is on the opposite side from the bone-facing surface. FIG. 8A is a side view and FIG. 8B is a plan view of the bone-facing surface 830.

The fixation elements in this embodiment are configured for an application where the metaphysical region of the glenoid is too small to receive multiples of more intrusive conventional fixation elements such as keels, posts, and pegs, etc. or even the multiple interference-fitting fixation elements 750 on the glenoid bearing implant embodiment 700, for example. For that reason, in the glenoid bearing implant 800 embodiment, generally there will be fewer fixation elements compared to the glenoid bearing implant embodiment 700. For example, in the example illustrated in FIGS. 8A-8B, only one interference-fitting fixation element 850 is provided. Arranged around the interference-fitting fixation element 850 closer to the perimeter of the bone-facing surface 830 are a plurality of smaller, less bulky, fixation elements 860 that present less intrusion into the metaphyseal region of the glenoid. These smaller fixation elements 860 can be monolithic spikes that are thinner and shorter than the interference-fitting fixation element 850. The interference-fitting fixation element 850 is structured the same as the interference-fitting fixation element 750 of the implant embodiment 700 and can be made of PEEK.

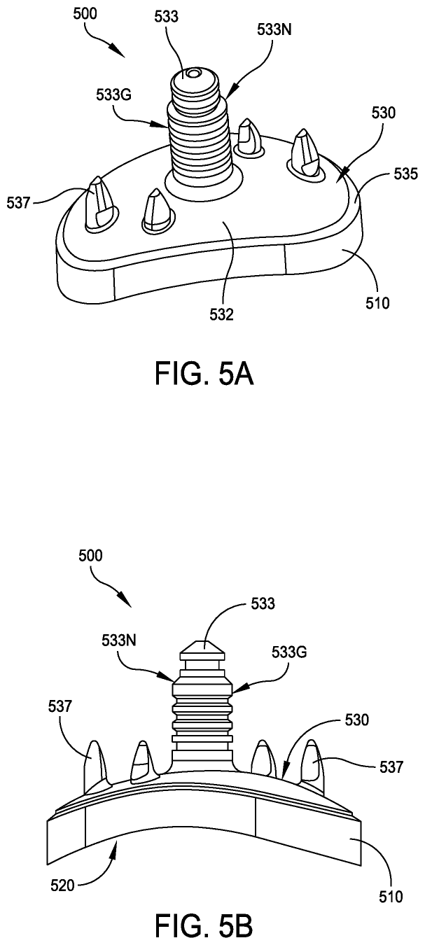

FIGS. 9A-9B show a glenoid bearing implant 900 according to another embodiment. The implant 900 comprises a bearing body 910 that is coupled to or molded onto a baseplate 915. The baseplate 915 comprises a plurality of fixation elements 950, 960 extending from the bone-facing surface 930 of the implant. An articulation surface 920 of the bearing body 910 is on the opposite side from the bone-facing surface 930. FIG. 9A is a side view and FIG. 9B is a plan view of the bone-facing surface 930.

The fixation elements 950, 960 in this embodiment are also configured for an application where the metaphyseal region of the glenoid is too small to receive multiples of more intrusive conventional fixation elements such as keels, posts, and pegs, etc. or even the multiple interference-fitting fixation elements 750 on the glenoid bearing implant embodiment 700, for example. The more centrally located fixation element 950 and the more peripherally located fixation elements 960 are smaller fixation elements that present less intrusion into the metaphysical region of the glenoid. These smaller fixation elements can be monolithic spikes similar to the fixation elements 860 of the implant embodiment 800. The baseplate 915 and the fixation elements 950, 960 can be formed of metal or PEEK, for example.

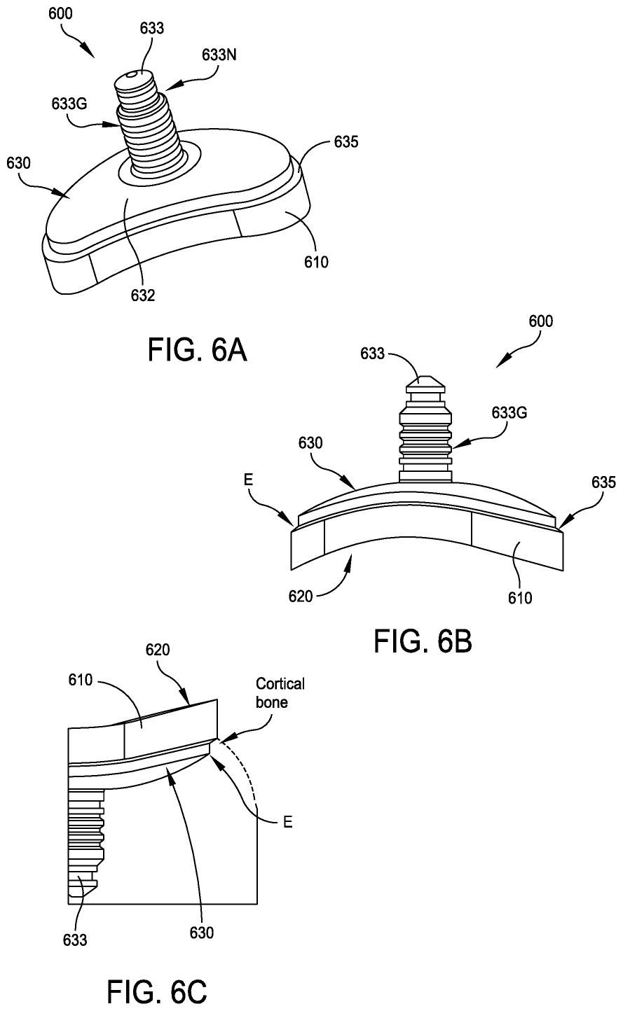

FIG. 10 shows a glenoid bearing implant 1000 according to another embodiment. The implant 1000 comprises a bearing body 1010, an articulation surface 1020, and a bone-facing surface 1030. Extending from the bone-facing surface 1030 are a plurality of fixation elements 1060 that can be elastically deformed during the implant procedure so that the fixation elements 1060 will apply static force against the glenoid bone and prevent the implant bearing body 1010 from rocking after implantation.

As shown, at rest, the fixation elements 1060 are in radially flared out configuration as shown. The fixation elements 1060 are formed of clastic material so that they can be elastically deformed (i.e. straightened out) to the disposition illustrated by the broken lines 1060′. For implantation, the glenoid surface is prepared appropriately by resecting or reaming and holes for receiving the fixation elements 1060 are drilled into the glenoid for receiving the fixation elements 1060. These holes are located and oriented in the glenoid such that the fixation elements 1060 must be temporarily pushed into the straight position as represented by the broken lines 1060′ from the at-rest position. The fixation elements 1060 are made from resilient material such as PEEK so that when they are pushed into the straight configuration and inserted into the prepared holes in the glenoid, they will try to spring back to their at-rest configuration and brace themselves against the sides of the holes and securely hold the implant 1000 in position thus preventing rocking motion.

Referring to FIGS. 11A-11B, disclosed is a configuration for asymmetrically arranged fixation elements for attaching a glenoid bearing implant or a baseplate to a glenoid when the strongest portion of the cancellous bone of the glenoid is not centrally (i.e., the geometric center of the glenoid) located. This means that the desired target anchoring region in the glenoid for the prosthesis is asymmetrically located. FIG. 11A is an illustration of a cross-sectional view of the glenoid (taken with the sectional plane being generally parallel to the articulating surface of the glenoid) showing an asymmetric location of the desired placement (i.e. the desired target anchoring region in the glenoid) for the fixation element for the glenoid bearing implant or a baseplate.

FIG. 11B is an illustration showing an embodiment of a glenoid implant 1100A having a primary fixation element 1133A with an outline of a glenoid for illustrating the asymmetric position of the primary fixation element 1133A. The glenoid implant 1100A has a body 1110A that may be a molded polymer bearing implant or a baseplate. The primary fixation element 1133A can be a porous metal keel or a post structure. To prevent rocking motion, the glenoid implant 1100A also comprises one or more shorter and smaller secondary fixation elements 1137A located away from the primary fixation element 1133A on the weaker side of the bone. The secondary fixation elements 1137A can be formed of poly or metal depending on the makeup of the body 1110A.

FIG. 11C is an illustration showing a glenoid implant 1100B according to another embodiment having a primary fixation element 1133B with an outline of a glenoid for illustrating the asymmetric position of the primary fixation element 1133B. The glenoid implant 1100B has a body 1110B that may be a molded polymer bearing implant. The bearing implant has an articulation surface 1120B. The primary fixation element 1133B can be a porous metal keel or a post structure. The body 1110B can have a metal portion 1115B that is co-molded with the polymer body 1110B and the primary fixation element 1133B extends from the metal portion 1115B. To prevent rocking motion, the glenoid implant 1100B also comprises one or more shorter and smaller secondary fixation elements 1137B located away from the primary fixation element 1133B on the weaker side of the bone. The secondary fixation elements 1137B can be formed of poly or metal depending on the makeup of the body 1110B.

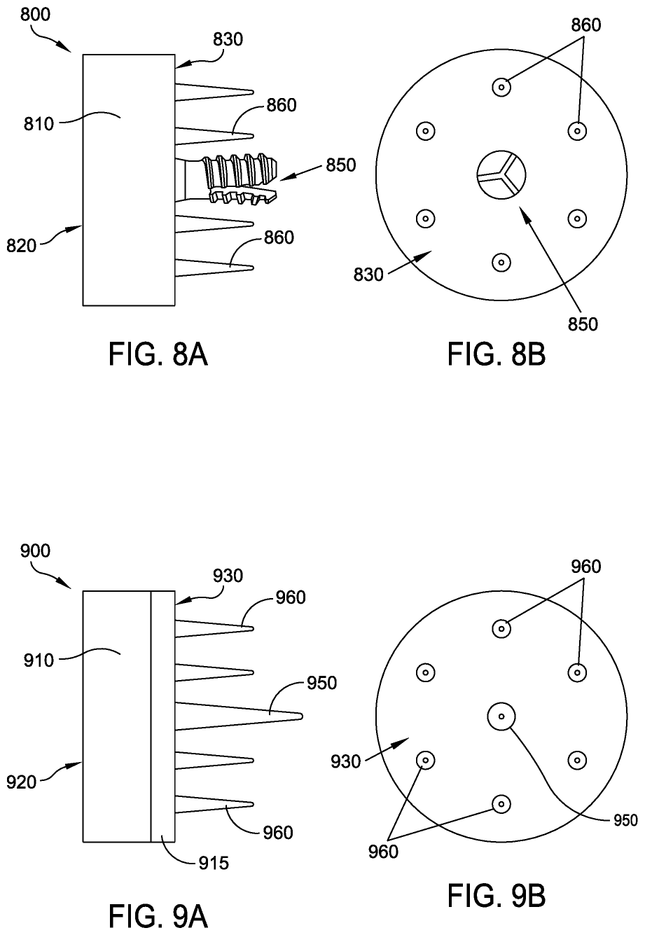

FIG. 12 shows a glenoid bearing implant 1200 according to another embodiment. The glenoid bearing implant 1200 comprises a bearing body 1210 having an articulation surface 1220 and a bone-facing surface 1230 on the opposite side of the body 1210. Extending from the bone-facing surface 1230 is an anchor post 1215. The distal end of the anchor post 1215 is split into two or more legs or ends 1215A, 1215B. The illustrated example has two legs 1215A and 1215B but some embodiments can have two or more. The bearing body 1210 has a fenestration 1217 that is aligned with the anchor post 1215 and provides a threaded hole that extends through the length of the anchor post 1215. When a screw 1240 is threaded into the fenestration 1217 from the articulation surface side as shown, as the screw 1240 extends into the anchor post 1215, the screw 1240 pushes the two or more legs 1215A, 1215B apart as noted by the arrows. This motion allows the glenoid bearing implant 1200 to be securely attached to a glenoid that has been prepared with a hole to receive the anchor post 1215.

The top end of the fenestration 1217 would be properly countersunk to accommodate the head of the screw 1240. As the fenestration 1217 is formed on the articulation surface 1220, the opening defining a rim of the fenestration 1217 is preferably chamfered to minimize the friction between the articulation surface 1220 and the opposing humeral head (anatomical or a prosthetic). In some embodiments, the chamfer angle can be about 45 degrees. The width of the chamfer is no greater than 1 mm to maximize the articulation surface area. In some embodiments, the chamfer is preferably between about 0.3-0.2 mm.

Additionally, the diameter of the fenestration 1217 on the articulation surface 1220 should not be too large as that would diminish the bearing function of the articulation surface 1220. The diameter of the hole also cannot be too small as it needs to accommodate the screw 1240 of sufficient diameter to operatively engage the anchor post 1215. The diameter of the fenestration 1217 can be optimized based on these parameters.

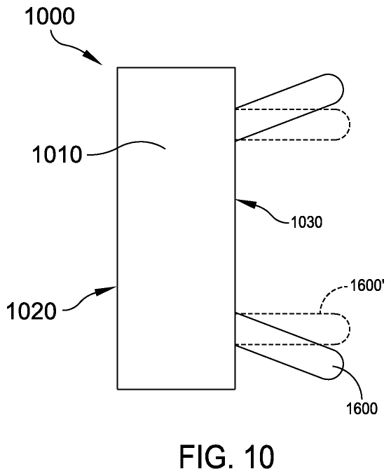

FIGS. 13A-13B are illustrations of a glenoid bearing implant 1300 according to another embodiment showing another configuration for a primary fixation of such implant. The glenoid bearing implant 1300 comprises a bearing body 1310 having an articulation surface 1320 and a bone-facing surface 1330 on the opposite side of the body 1310. Extending from the bone-facing surface 1330 is an anchor post 1315 that is cannulated to receive and accommodate a screw 1340 through the length of the anchor post 1315. The bearing body 1310 has an opening 1317 positioned aligned with the cannulated anchor post 1315 allowing the screw 1340 to be inserted into the cannulated anchor post 1315 from the articulation surface 1320 and penetrate through the length of the anchor post 1315. The channel inside the cannulated anchor post 1315 is illustrated by the broken lines 1315E. The opening 1317 is appropriately countersunk to prevent the head of the screw 1340 from traveling through the bearing body 1310.

The anchor post 1315 also has an elongated opening 1315A that extends along a portion of the length of the anchor post 1315 and extending laterally through the shaft of the anchor post 1315, thus, dividing the shaft of the anchor post 1315 into two portions 1315B and 1315C.

The internal surface of the channel 1315E is threaded at the distal end portion 1315D to enable the distal end portion 1315D to threadedly engage the screw 1340. With this configuration, when the screw 1340 is inserted into the anchor post 1315 from the articulation surface side and the distal end of the screw threads into the distal end portion 1315D of the anchor post 1315 and the head of the screw bottoms out in the countersink of the opening 1317, continuously turning the screw 1340 in the threading direction will cause the threaded distal end portion 1315D of the anchor post 1315 to be pulled in the direction toward the head of the screw and the bearing body 1310. This motion will cause the two portions 1315B and 1315C of the anchor post 1315 to bulge radially outward as shown in FIG. 13B. This actuation mechanism can be used to securely anchor the glenoid bearing implant 1300 into a glenoid that is prepared with a hole to receive the anchor post 1315.

In use, after the patient's glenoid is prepared to receive the glenoid bearing implant 1300, the anchor post 1315 of the glenoid bearing implant 1300 is inserted into a hole thus prepared in the patient's glenoid. Then, the screw 1340 is inserted through the opening 1317 and into the channel 1315E until the screw 1340 engages the distal end portion 1315D of the anchor post. Then, by turning the screw 1340 to thread into the distal end portion 1315D and pulling the distal end portion 1315D in the direction toward the bearing body 1310, the anchor post 1315 is urged into the configuration shown in FIG. 13B securing the implant 1300 in place.

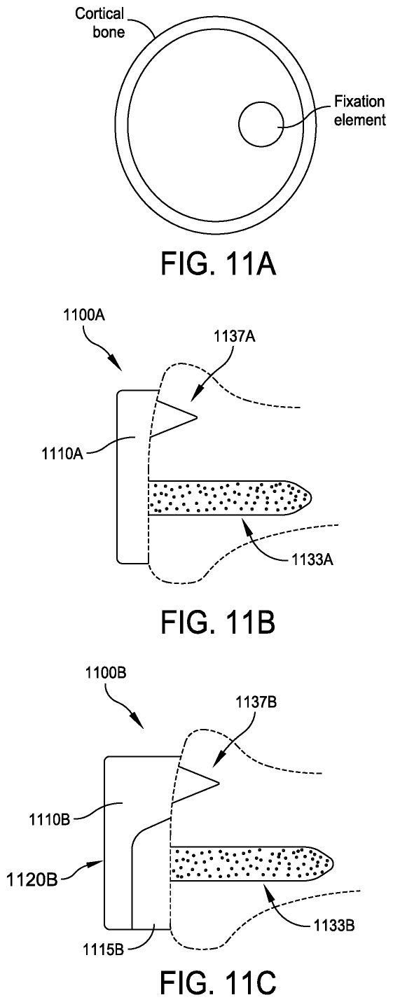

FIGS. 14A-14B are illustrations of a glenoid bearing implant 1400 according to another embodiment. The implant 1400 comprises a bearing body 1410 having an articulation surface 1420, a bone-facing surface 1430, and an anchor post 1415 extending from the bone-facing surface 1430. The anchor post 1415 has an elongated opening 1415A that extends along a portion of the length of the anchor post 1415 and extending laterally through the shaft of the anchor post 1415 thus dividing the shaft of the anchor post 1415 into two portions 1415B and 1415C in the middle region of the anchor post 1415 between the bearing body 1410 and the distal end 1415D of the anchor post. This configuration is similar to that of the bearing implant embodiment 1300. Similar to the bearing implant embodiment 1300, pulling the distal end 1415D of the anchor post toward the bearing body 1410 causes the two portions 1415B and 1415C of the anchor post to bulge radially outward as shown in FIG. 14B. This actuation mechanism can be used to securely anchor the glenoid bearing implant 1400 into a glenoid that is prepared with a hole to receive the anchor post 1415. In the embodiment 1400, although not shown in FIGS. 14A and 14B, the pulling action of the distal end 1415D can be accomplished with an appropriate instrument rather than a screw as in the embodiment 1300.

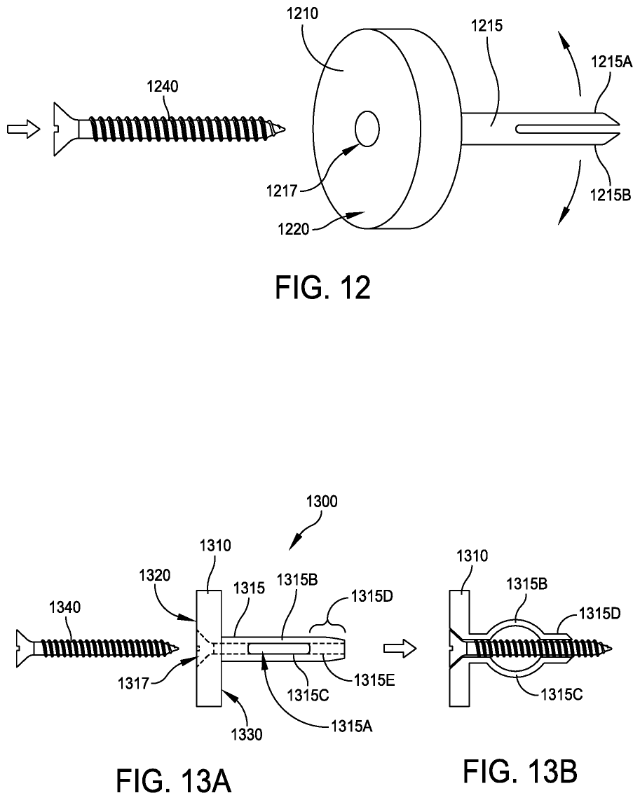

FIG. 15 is an illustration of a fixation element configuration 1500 for achieving enhanced primary fixation of a baseplate implant to a glenoid. In this configuration, two or more fixation elements such as the two screws 1540 shown are used to secure a baseplate implant 1510 to a prepared glenoid G. To minimize the rocking motion of the baseplate implant 1510, the implant trajectory of the screws 1540 should be angled toward the central axis A of the glenoid so that the trajectories of the screws crossover as shown. This arrangement of the two screws 1540 help prevent or minimize rocking motion of the baseplate implant 1510 represented by the arrows R.

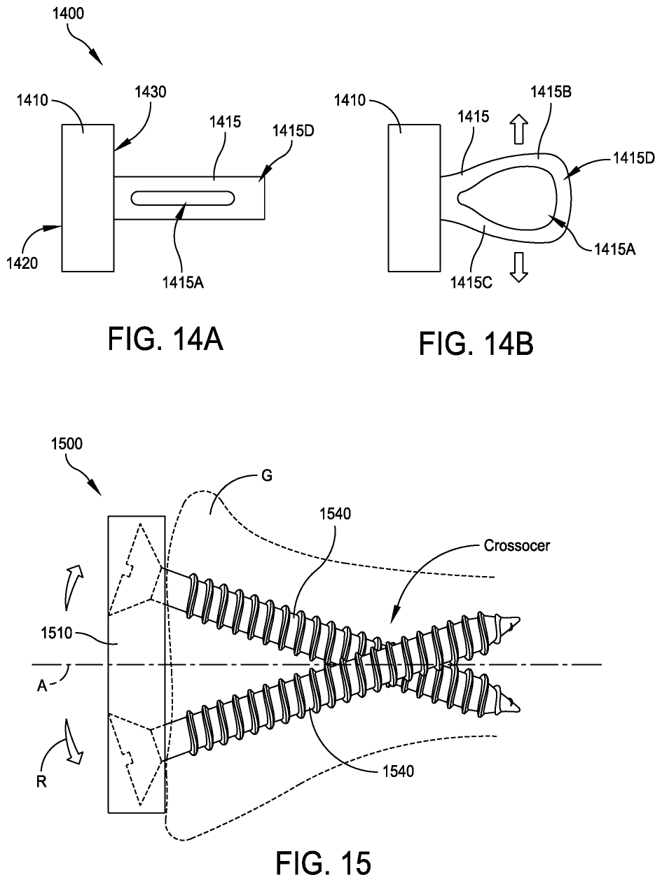

FIG. 16 is an illustration of another fixation element configuration for achieving enhanced primary fixation of a glenoid bearing implant 1600. The glenoid bearing implant 1600 comprises a bearing body 1610, an articulation surface 1620, a bone-facing surface 1630 and an anchor post 1615. Similar to the anchor posts in the implant embodiments 1300 and 1400, the anchor post 1615 is configured with an elongated opening 1615A that extends along a portion of the length of the anchor post 1615 and extending laterally through the shaft portion of the anchor post 1615. In this embodiment, however, the bulging or expansion of the portion of the anchor post 1615 near the opening 1615A is accomplished by threading a screw 1640 through the opening 1615A from the side of the glenoid after the anchor post 1615 is inserted into a hole prepared into the glenoid. The screw 1640 has diameter larger than the width of the opening 1615A so that as the screw 1640 threads into the opening 1615A it deforms and expands the middle portion of the anchor post 1615A.

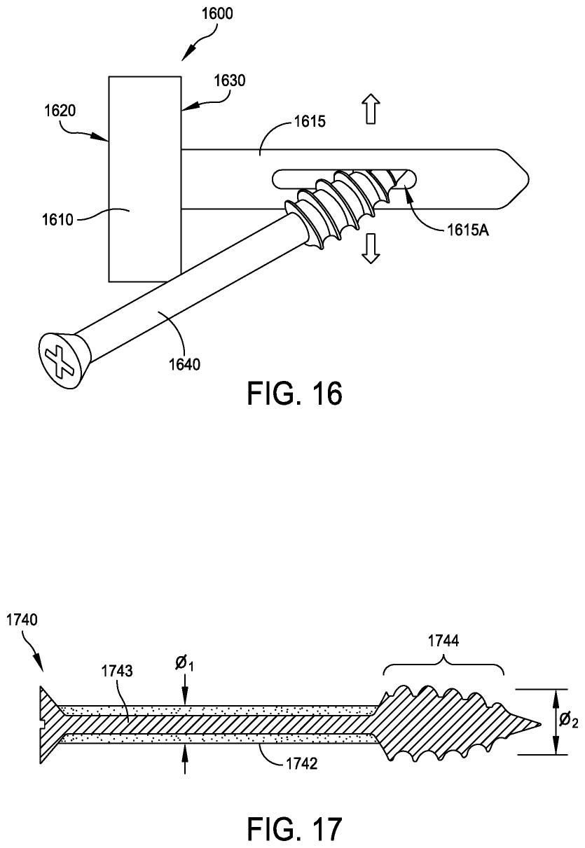

FIG. 17 is an illustration of a new bone screw construct 1740 that would be useful in any orthopedic applications where a screw is extending through cancellous bone region to thread into a cortical bone. The bone screw 1740 comprises a threaded tip region 1744 and a porous shaft region 1742 between the screw head and the threaded tip region 1744. The porous shaft region 1742 has a solid core 1743. The diameter of the porous shaft region is denoted as ϕ₁ and the diameter of the threaded tip region 1744 is denoted as ϕ₂ and ϕ₂<ϕ₁. Such screw can be useful in glenoid implant applications where the glenoid exhibits erosion in the subchondral bone and the bone screw 1740 can be used to achieve cortical fixation through the subchondral bone region.

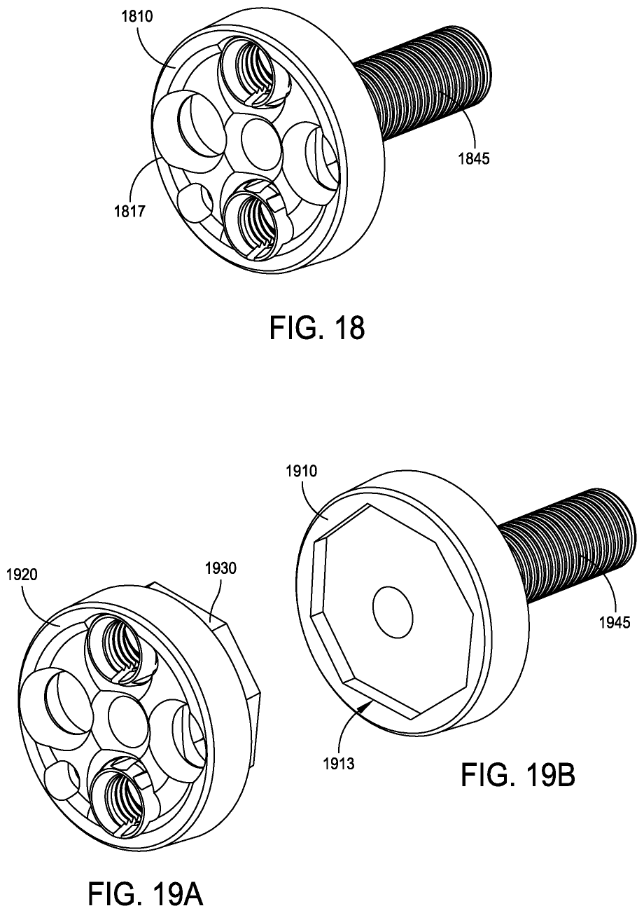

FIG. 18 is an illustration of an embodiment of a baseplate 1810 for reverse shoulder arthroplasty (RSA) application. The baseplate 1810 comprises a threaded anchor post 1845 extending from the bone-facing side of the baseplate 1810 and a plurality of holes 1817 for bone screws to provide additional fixation. The threaded anchor post 1845 provides the primary fixation of the baseplate 1810 to a glenoid. The whole baseplate 1810 can be turned to thread the threaded anchor post 1845 into a glenoid.

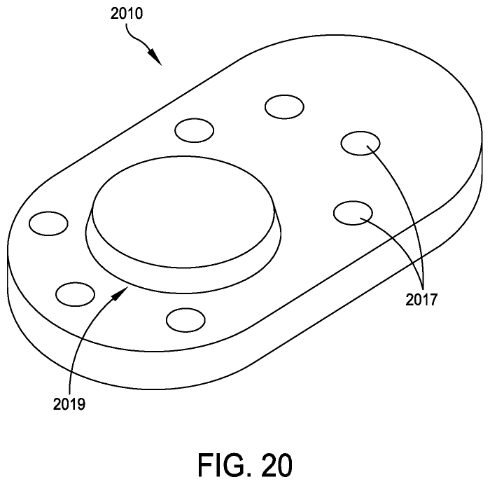

FIGS. 19A-19B are illustrations of a baseplate 1910 according to another embodiment for RSA application. The baseplate 1910 comprises a threaded anchor post 1945 extending from the bone-facing surface of the baseplate 1910. The baseplate 1910 comprises a polygon shaped recess 1913 provided on the surface opposite from the bone-facing surface for receiving a glenosphere component 1920, i.e. a convex articulation component. The glenosphere component 1920 is configured with a base 1930 that has a polygon shaped outline that corresponds to the polygon shape of the recess 1913. The polygon shaped outlines enhance the torsional stability of the glenosphere.

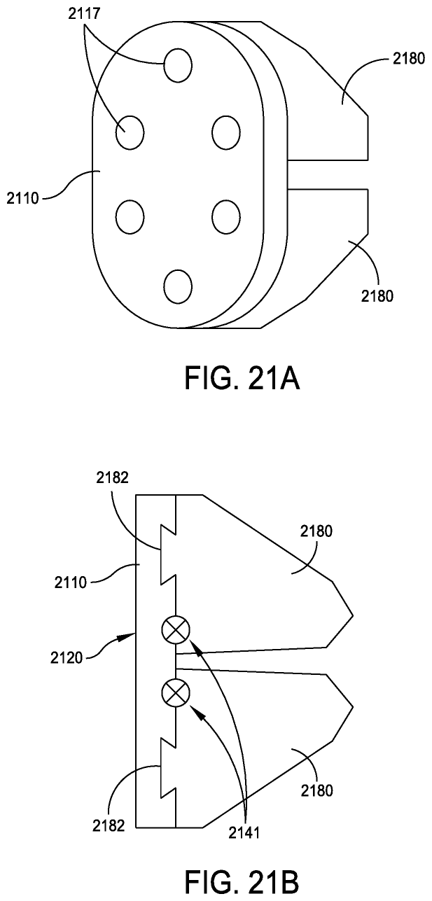

FIG. 20 is an illustration of baseplate 2010 according to another embodiment for RSA applications. The baseplate 2010 comprises a plurality of screw holes 2017 for securing the baseplate to a glenoid. The baseplate 2010 further comprises a conical boss 2019 for attaching a corresponding anatomical glenoid bearing implant component (not shown) or a glenosphere component (not shown). The glenoid bearing implant component and the glenosphere component would be configured with a conical recess that complements the conical boss 2019 to achieve a secure attachment. The conical boss and the conical recess' mating surfaces can be Morse taper surfaces.

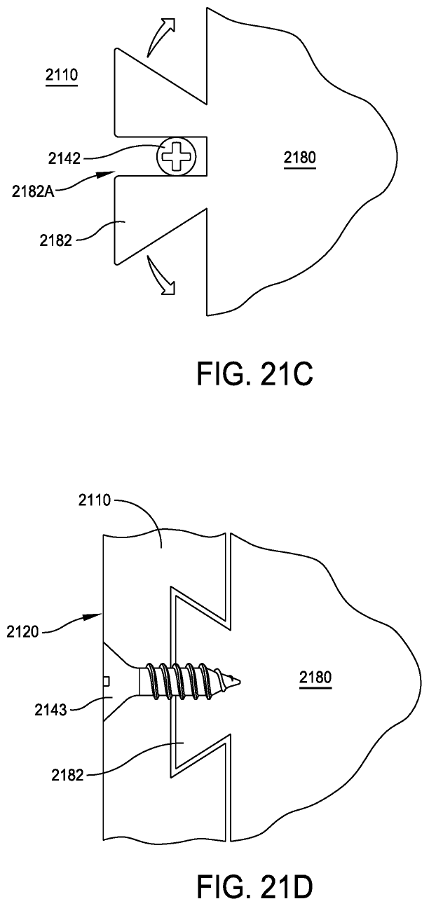

Reverse augmented baseplates are used for treatment of glenoid bone wear conditions in RSA applications. FIGS. 21A-21D are illustrations of a baseplate 2110 configured as a system utilizing modular augments 2180 for RSA application. The baseplate 2110 includes a plurality of holes 2117 for receiving bone screws for securing the baseplate 2110 to a glenoid. The bone-facing surface side of the baseplate 2110 is configured for receiving and securing thereto one or more modular augments 2180. FIG. 21B shows a sectional side view of the baseplate 2110 with two such modular augments 2180 attached.

Each of the modular augments 2180 and the baseplate 2110 are configured to be coupled together by one or more dovetail structures 2182 as shown by sliding into each other. In the illustrated example, the augments 2180 are configured with the male part of the dovetail mechanism and the baseplate 2110 is configured with the corresponding female part of the dovetail mechanism. In the example shown in FIG. 21B, after the augments 2180 are coupled to the baseplate 2110, each augment 2180 can be securely locked in position by a set screw 2141.

In another example shown in FIG. 21C, a set screw 2142 is inserted through the dovetail 2182 to securely lock the augment 2180 to the base plate 2110. In this example, the dovetail 2182 structure on the augment has a slot 2182A in the middle of the dovetail dividing the dovetail 2182 into two portions and the set screw 2142 is driven into position into the slot 2182A from the side through the baseplate 2110. The set screw 2142 has a diameter that is larger than the width of the slot 2182A such that as the set screw 2142 is driven into the slot 2182A, the set screw pushes the two portions of the dovetail 2182 outward as shown and urges the two portions of the dovetail 2182 into the corresponding complementary surfaces of the baseplate 2110 and securely lock the augment 2180 to the baseplate 2110.

In another example shown in FIG. 21D, a set screw 2143 is inserted through the dovetail 2182 from the top surface 2120 side of the baseplate 2110 to thread into the dovetail 2182 pulling the dovetail 2182 and the baseplate 2110 together tight. This direction would be orthogonal to the direction of the set screw 2142 insertion shown in FIG. 21C.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

1. A prosthetic implant for glenoid comprising:
a baseplate configured to be anchored to a bone, the baseplate comprises:
a bone-facing surface; and
a second surface opposite the bone-facing surface, wherein the second surface is configured with a pair of slots for receiving and releasably coupling with a corresponding pair of parallel tabs provided on an articulating component.

2. The prosthetic implant of claim 1, wherein the baseplate has a superior end and an inferior end corresponding to the glenoid's anatomical directions, with a median line defined halfway between the superior end and the inferior end, wherein the slot located on the superior end side of the median line is located closer to the superior end than the median line and the slot located on the inferior end side of the median line is located closer to the inferior end than the median line.

3. The prosthetic implant of claim 2, wherein each of the pair of slots are straight and they are parallel to the median line.

4. The prosthetic implant of claim 2, wherein each of the pair of slots have an outline with a curvature.

5. The prosthetic implant of claim 4, wherein the baseplate comprises a rim along the periphery of the second surface that extends away from the bone-facing surface and the curvature of the slot located on the superior end side of the median line is substantially parallel to the curvature of the rim along the superior end of the baseplate and the curvature of the slot located on the inferior end side of the median line is substantially parallel to the curvature of the rim along the inferior end of the baseplate.

6. The prosthetic implant of claim 4, wherein the curvature of the slot located on the superior end side of the median line has a radius of curvature that is not less than radius of curvature of the rim along the superior end, and the curvature of the slot located on the inferior end side of the median line has a radius of curvature that is not less than radius of curvature of the rim along the inferior end.

7. The prosthetic implant of claim 3, wherein the baseplate has a superior end and an inferior end corresponding to the glenoid's anatomical directions, with a median line defined halfway between the superior end and the inferior end and the median line is parallel to the pair of parallel slots;
wherein one of the pair of slots is located on the superior end side of the median line and the other of the pair of slots is located on the inferior end side of the median line.

8. The prosthetic implant of claim 1, wherein each of the pair of slots has an L-shaped a cross-section having an elbow portion, wherein the elbow portion of the L-shape pointing away from the other slot of the pair of slots.

9. A prosthetic implant for glenoid comprising:
a bearing component that comprises a bearing surface, a baseplate-facing surface, and a pair of parallel tabs provided on the baseplate-facing surface; and
a baseplate configured to be anchored to a bone, and comprising a bone-facing surface and a second surface opposite the bone-facing surface, wherein the second surface is configured with a pair of parallel slots for receiving and engaging with the corresponding pair of parallel tabs for releasably coupling the bearing component to the baseplate.

10. The prosthetic implant of claim 9, wherein the baseplate has a superior end and an inferior end corresponding to the glenoid's anatomical directions, with a median line defined halfway between the superior end and the inferior end and the median line is parallel to the pair of parallel slots;
wherein one of the pair of parallel slots is located on the superior end side of the median line and the other of the pair of parallel slots is located on the inferior end side of the median line.

11. The prosthetic implant of claim 10, wherein the parallel slot located on the superior end side of the median line is located closer to the superior end than the median line and the parallel slot located on the inferior end side of the median line is located closer to the inferior end than the median line.

12. The prosthetic implant of claim 10, wherein the bearing component is configured so that one of the pair of parallel tabs on the bearing component forms a snap-fit engagement with the corresponding one of the pair of parallel slots on the baseplate.

13. The prosthetic implant of claim 12, wherein said tab that forms a snap-fit engagement is the one corresponding to the slot that is located on the superior end side of the median line on the baseplate.

14. The prosthetic implant of claim 3, wherein the baseplate has an anterior side and a posterior side corresponding to the glenoid's anatomical directions, wherein the pair of parallel slots on the baseplate are open to one of said two sides of the baseplate such that the bearing component can be releasably coupled to the baseplate by sliding the corresponding pair of parallel tabs on the bearing component into the pair of parallel slots from the open side.

15. The prosthetic implant of claim 14, wherein the baseplate-facing surfaces of the bearing component and the second surface of the baseplate are configured to form an interference fit when the bearing component is fully seated in the baseplate, thereby preventing the bearing component from unintentionally being disengaged.

16.-40. (canceled)
